(19) United States
(12) Patent Application Publication
(10) Pub. No.: US 2024/0139372 A1
(43) Pub. Date: May 2, 2024

(54) SYSTEMS, APPARATUSES, AND METHODS FOR APPLYING A COATING LAYER TO A MEDICAL IMPLANT

(71) Applicant: Abbott Cardiovascular Systems, Inc, Santa Clara, CA (US)

(72) Inventors: Ni Ding, San Jose, CA (US); Vincent James Gray, Clonmel (IE)

(21) Appl. No.: 18/450,941

(22) Filed: Aug. 16, 2023

Related U.S. Application Data
(60) Provisional application No. 63/419,504, filed on Oct. 26, 2022.

(51) Int. Cl. A61L 27/18; A61L 27/54

(52) U.S. Cl. CPC A61L 27/18; A61L 27/54; A61L 2420/06

(57) ABSTRACT

Systems, methods, and apparatuses for depositing polymer and/or other layers onto surfaces of, for example, implantable medical devices. In some embodiments, the polymer and/or other coating layers are deposited via plasma polymerization deposition. In some embodiments, primer layers are deposited via plasma polymerization deposition. A coating layer can be formed over a primer layer. The deposited layers can be bioresorbable and/or bioabsorbable.

### CROSS-REFERENCE TO RELATED APPLICATIONS

This application claims the benefit of and priority to U.S. Provisional Patent Application Ser. No. 63/419,504 filed on Oct. 26, 2022, and entitled “SYSTEMS, APPARATUSES, AND METHODS FOR APPLYING A COATING LAYER TO A MEDICAL IMPLANT,” which application is expressly incorporated herein by reference in its entirety.

### BACKGROUND OF THE INVENTION

### 1. The Field of the Invention

The present disclosure relates generally to systems and apparatuses for medical implants having bioresorbable coatings, and methods for the manufacture and use thereof.

### 2. The Relevant Technology

Many medical devices use coatings to facilitate the delivery of drugs and/or other therapeutic agents to target anatomies within patients. For example, stents that are left in a patient to help keep blood vessels open may also be coated with therapeutic agents to treat a target anatomy (e.g., the blood vessel) while implanted within the patient.

These drug containing coatings generally do not adhere well to metals; a base material from which many medical implants are constructed. Thus, to adequately adhere these coatings to a medical implant, a primer layer is typically applied to the implant first. The drug coating can then be applied to the primer, to which the coating adheres well.

Current methods of applying a primer to a medical implant typically apply thick layers. For example, methods such as dip- or spray-coating generally apply primer layers of approximately 1-2 μm. The thickness of the applied primer layer can impact performance of the medical implant. A thicker primer coating contributes to the total coating profile of the medical implant. Thicker layers (i.e., thicker coating profiles) may promote cracking or delamination of both the primer and coating layers. This may lead to particulate shedding and embolization and insufficient delivery of drugs and/or therapeutic agents to the target vessel.

### BRIEF DESCRIPTION OF THE DRAWINGS

To further clarify the above and other advantages and features of the present invention, a more particular description of the invention will be rendered by reference to specific embodiments thereof which are illustrated in the appended drawings. It is appreciated that these drawings depict only illustrated embodiments of the invention and are therefore not to be considered limiting of its scope. The invention will be described and explained with additional specificity and detail through the use of the accompanying drawings in which:

FIGS. 1A and 1B illustrate a system for depositing a layer on a surface of a medical device, according to one embodiment of the invention.

FIG. 2 illustrates a medical device or implant having a primer layer and a coating layer, according to one embodiment of the invention.

FIG. 3 illustrates one example of a monomer to be used in a primer layer and/or coating layer, according to one embodiment of the invention.

FIG. 4 illustrates a flowchart of an example method for depositing a layer, according to one embodiment of the invention.

FIG. 5 schematically illustrates an example of the plasma polymerization and deposition process according to the present disclosure.

FIGS. 6-7B illustrate SEM images of coating layers applied to a substrate, such as a medical implant, according to embodiments of the present disclosure.

### DETAILED DESCRIPTION OF THE INVENTION

One or more specific embodiments of the present disclosure will be described below. In an effort to provide a concise description of these embodiments, some features of an actual embodiment may be described in the specification. It should be appreciated that in the development of any such actual embodiment, as in any engineering or design project, numerous embodiment-specific decisions will be made to achieve the developers' specific goals, such as compliance with system-related and business-related constraints, which may vary from one embodiment to another. It should further be appreciated that such a development effort might be complex and time consuming, but would nevertheless be a routine undertaking of design, fabrication, and manufacture for those of ordinary skill having the benefit of this disclosure.

One or more embodiments of the present disclosure may generally relate to forming a layer on a medical implant, such as but not limited to a metallic or polymeric implantable device. Additionally, the one or more embodiments of the present disclosure also relate to forming a layer on a medical implant, such as expandable or self-expanding scaffolds, stents, filters, temporary implants, permanent implants, etc. Additionally, the one or more embodiments of the present disclosure also relate to forming a primer layer on a medical implant, such as expandable or self-expanding scaffolds, stents, filters, temporary implants, permanent implants, etc. Additionally, the one or more embodiments of the present disclosure are also related to forming a coating layer on a medical implant, such as expandable or self-expanding scaffolds, stents, filters, temporary implants, permanent implants, etc., with or without forming a primer layer on the medical implant before forming the coating layer.

Additionally, the one or more embodiments of the present disclosure may generally relate to apparatuses, systems, and methods for depositing a layer on a medical implant, such as a primer layer, a coating layer on the primer layer, one or more coating layers on the coating layer deposited on the primer layer, combinations or modifications thereof. The apparatuses, systems, and methods can also be associated, more generally, with polymerization reactions, such as free radical ring opening polymerization, chain polymerization, plasma polymerization, etc.

While the present disclosure will describe a particular implementation of apparatuses and systems, with associated methods, for depositing primer layers on medical implants, it should be understood that any of systems, apparatuses, and methods described herein may be applicable to other uses, including and not limited to depositing thin layers (e.g., thin coating layers) on medical devices having coatings or other films; deposition of any polymer layer on an implantable medical device, with or without a previously deposited layer; etc. Additionally, elements described in relation to any embodiment depicted and/or described herein may be combinable with elements described in relation to any other embodiment depicted and/or described herein.

While embodiments of the present disclosure are discussed with relation to a particular set of ringed monomers, it is to be understood that other appropriate ring monomers can be utilized with methods and systems of the present disclosure. The particular ring monomers chosen will be influenced by the resulting application for the layers, such as primers and other coating layers produced according to embodiments of the present disclosure.

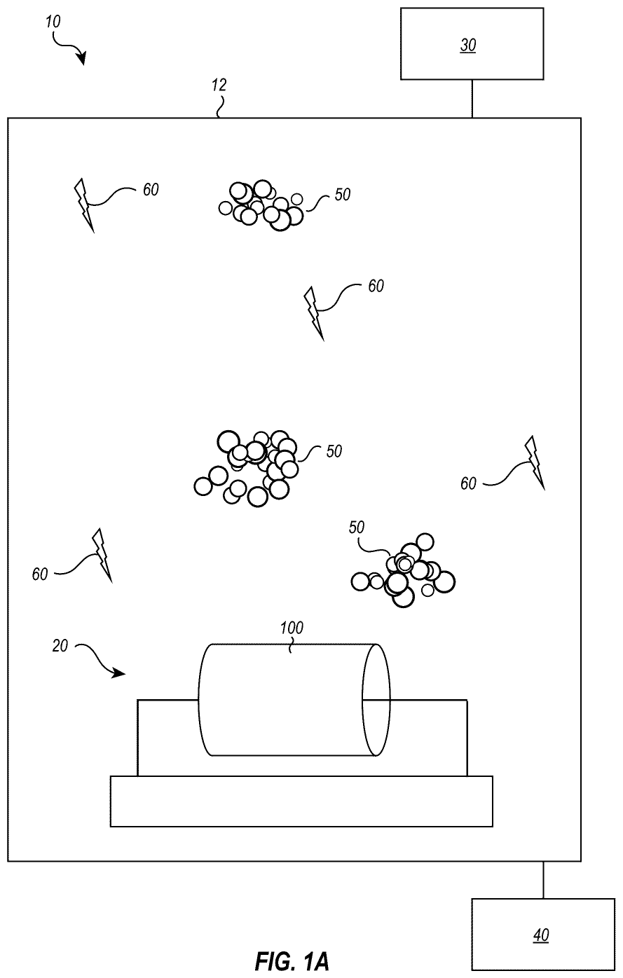

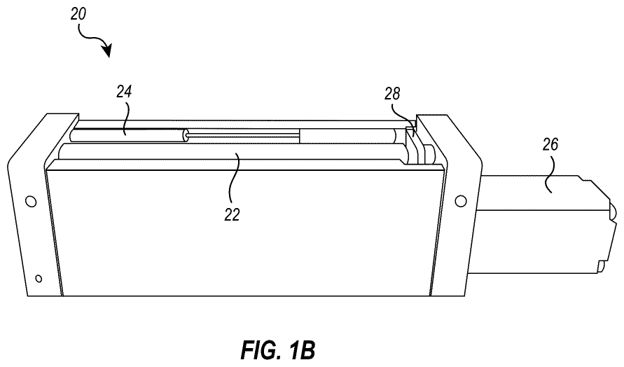

FIGS. 1A-1B illustrate a system 10 for depositing a layer on at least one surface of a medical device, according to one embodiment of the invention. The layer deposited on at least one surface of the medical device has a thickness significantly thinner than current approaches to forming a layer, such as a primer layer that improves adhesion between a body or structure of the medical device, such as a drug-containing bioresorbable scaffold (BRS) and other layers contained or included with the medical device, such as coatings with therapeutic agents, etc. Some drug-containing bioresorbable scaffolds (BRS) contain a poly (D,L-lactide) (PDLLA) and everolimus coating. Other medical devices, such as a metal stent, such as a Cobalt-Chromium (Co—Cr) stent or other metallic stent can be coated with a coating including poly (D,L-lactide) (PDLLA) and everolimus. Application of such a coating directly onto such a metal stent, would result in poor adhesion of the coating to the medical implant. This poor adhesion is likely due to, at least, two factors:
1) the glass transition temperature (T_{g}) of PDLLA is 56° C. Both at room temperature and 37° C., PDLLA is a relatively brittle polymer. Crimping and expansion of the scaffold compresses and stretches the coating, resulting in coating fracture and delamination; and
2) high drug-to-polymer ratio (e.g., 1:1) contributes to poor adhesion, as everolimus has poor adhesion to metal scaffold.

According to embodiments of the present disclosure, layers, such as primer layers or other layers, including a plurality of monomers can be polymerized directly on medical implants (e.g., surfaces thereof). This can result in layer thicknesses that are significantly thinner than current approaches provide in forming a layer, such as a primer layer. The thickness of the deposited layer can range from 5-100 nm for a polymer, as will be described in more detail herein. It will be understood that the layer thicknesses can range from about 5 nm to about 500 nm for a polymer, from about 7 nm to about 100 nm for a polymer, from about 10 nm to about 50 nm for a polymer, or from about 10 nm to about 20 nm for a polymer.

As mentioned above, applying the primer layer can improve adhesion of a subsequently deposited coating or layer. Bioresorbable polymers such as poly (L-lactide) (PLLA), poly(D,L-lactide (PDLLA), polycaprolactone (PCL), polyglycolide (PGA), poly(trimethylene carbonate), polydioxanone, or combination of these ring monomers, are often synthesized via ring opening polymerization (ROP) and/or polycondensation reactions, while many vinyl or acrylate-based polymers (such as PBMA and PVDF-HFP) can be synthesized by free radical polymerization. While reference is made to deposition of a primer layer onto the medical implant using ring opening polymerization (ROP) and/or polycondensation reactions, free radical ring open polymerization (RROP) often requires free radical initiator for RROP reaction. As discussed below, plasma polymerization requires no chemical initiator for such polymerization.

The monomers and resultant polymers that can be applied or deposited can include, but not limited to, natural bioresorbable or bioabsorbable polymers, synthetic bioresorbable or bioabsorbable polymers, such as Polyglycolic Acid (PGA), Polylactic Acid (PLA), Poly (ε-caprolactone) (PCL), polydioxanone, random copolymer, block copolymer, combinations or mixtures thereof. Additionally, for those monomers that are not liquids, the monomer can be dissolved in a solvent, such as aqueous solvent, organic solvent, and/or other liquid monomer substrates. For instance, in some embodiments, a solid monomer such as (D,L-lactide) DLLA can be dissolved in caprolactone (CL), where CL is a monomer and also a solvent. In other configurations, the monomer can be dissolved in a solvent for plasma polymerization, where the solvent can also include other liquid monomers, such as CL, dioxanone (>30C), and other liquid monomers.

In some embodiments, the primer layer for example, of the present disclosure includes copolymers. For example, copolymers can be polymerized directly on a medical implant via plasma deposition polymerization. In some embodiments, the copolymers include PDLLA and polycaprolactone (PCL). In some embodiments, the ratio of PDLLA to PCL ranges from approximately 100:0 to 0:100, such as 1:9, 3:7, 1:1, 7:3, 9:1, etc. Since DLLA is a solid, solvent such as acetone, chloroform, ethanol, isopropanol, other aqueous solvents, organic solvents, other liquid monomer substrates, etc. may be used before mixing with CL. At higher CL ratio, DLLA can be dissolved in CL. In some embodiments, the copolymers further include poly (L-lactide) (PLLA). In some embodiments, the ratio of PDLLA to other copolymers in the primer layer ranges from 100:0 to 0:100, such as 1:9, 3:7, 1:1, 7:3, 9:1, etc. In some embodiments, the ratio of PLLA to other copolymers in the primer layer ranges from 100:0 to 0:100, such as 1:9, 3:7, 1:1, 7:3, 9:1, etc.

In some embodiments, the medical implant can be constructed, at least partially, from a polymer, such as PLLA. Due to the polymer structural similarity of a PDLLA/everolimus drug coating and a PLLA medical implant, a coating such as PDLLA/everolimus can be directly deposited on the medical implant using the methods and systems described herein, or otherwise contemplated by the disclosure, to form a drug-containing bioresorbable scaffolds.

In some embodiments, the medical implant is constructed, at least partially, from a metal. Due to the dissimilarities in structure between the metal implant and, for example, a PDLLA/everolimus drug coating, the adhesion of the PDLLA/everolimus coating may be inferior and requires an intervening primer layer. While reference is made to applying a PDLLA/everolimus drug coating using plasma polymerization, plasma polymerization can be used with other polymer-drug coatings (e.g., where the therapeutic agent includes any of a variety of olimus-based drugs, such as, but not limited to everolimus, rapamycin, zotarolimus, biolimus, and/or novolimus. For example, a coating polymer that is deposited directly on a medical implant body or on a primer layer can be a copolymer of PGA-PDLLA, PDLLA-PCL, PGA-PCL, P(GA-CL-TMC, P(LLA-CL-TMC), P(DLLA-CL-TMC), etc. In some embodiments, the primer layer is PDLLA. In some embodiments, the primer layer is PCL. In some embodiments, the primer layer includes a mixture of PDLLA and PCL. More generally, the primer layer can have a similar composition to the composition of a coating to be deposited thereupon without inclusion of the therapeutic agent. For instance, when a scaffold is to be coated with polymer A mixed with a therapeutic agent, the primer layer can include polymer A or a combination or mixture containing polymer A.

Shown in FIG. 1A and 1B is a system 10 for use in depositing a layer onto a medical device. The system 10 includes a chamber 12, within which is placed a medical implant or device 100. The medical implant or device 100 can be supported by support assembly 20 so that the different surfaces of the medical implant or device 100 can be exposed to the gas plasma within the chamber 12. In one configuration, shown in FIG. 1B, the support assembly 20 can include rollers 22 upon which is disposed a medical device holder 24. The rollers 22 are connected to a motor 26 through various gears, linkages 28, etc. so that rotation or movement of the motor 26 rotates the medical device holder 24 and so the medical implant or device 100 supported by the medical device holder 24.

Returning to FIG. 1A, as mentioned above, the system 10 includes the chamber 12 that contains a gas or mixture of gases, such as argon, nitrogen, etc. and monomers used to deposit the coating upon the medical implant or device 100. The particular plasma polymerization process and system can vary based upon particular design and apparatus selected for the deposition. For instance, the present disclosure and system 10 can be a static-type chamber or reactor system, a continuous flow chamber or reactor system, an electrodeless chamber or reactor system, or any other plasma polymerization chamber or reactor system. As such, the system 10 can optionally include a gas supply 30 that allows gas and/or monomer solution 50 to flow continuously into the interior of the chamber 12 or can allow a controlled amount of gas and/or monomer solution 50 into the chamber 12.

With the monomer solution 50 and gas within the chamber 12, high voltage from the power supply 40 can activate the precursors to generate a gas plasma, including free radicals 60. The power supply 40 can utilize radiofrequency coils disposed around the chamber 12 or can include electrodes disposed within the chamber 12 to activate the gas and monomer solution 50 to generate the free radicals 60. For instance, the free radicals 60 can be generated from a gas plasma, where the gas plasma includes one or more inert gases. For example, the gas plasma can include argon, nitrogen, and/or combinations thereof. In some embodiments, the gas plasma may additionally include other inert or reactive gases, such as neon, atmospheric air, ammonium and/or combinations or mixtures thereof.

The free radicals 60 interact with the monomer solution 50. For example, the free radicals 60 open the monomers rings 50. Opening of the monomeric ring starts the chain propagation and chain growth. The polymer chain is deposited directly on a surface of the medical implant or device 100. Additionally, and/or alternatively, monomers from the monomer solution may be deposited onto the surface of the medical implant or device 100 and polymerize with other monomers directly on the surface of the medical implant or device 100.

Plasma polymerization has an advantage over traditional synthetic methods for producing coatings (e.g., the primer layer or other layers) directly on the medical device. For example, instead of using free radical initiators or heavy metal catalysts to initiate the polymerization, gas plasma (e.g., gases or mixtures of gases, such as nitrogen, argon, etc. with an applied voltage) acts as an initiator to polymerize the vinyl or acrylate polymer and in this case, free radical ring opening polymerization. This approach introduces no terminal groups from the gas plasma initiator and uses no catalyst.

Further, plasma polymerization beneficially results in the deposition of nanometer thin coatings in a controlled, uniform manner. That is, the coatings are deposited more uniformly on the medical implant in comparison to current deposition methods for medical implants, and the coating thickness is tunable. Additionally, there is no need to subsequently dry the primed medical implants as the solvents used in the monomer solution evaporate during the deposition process.

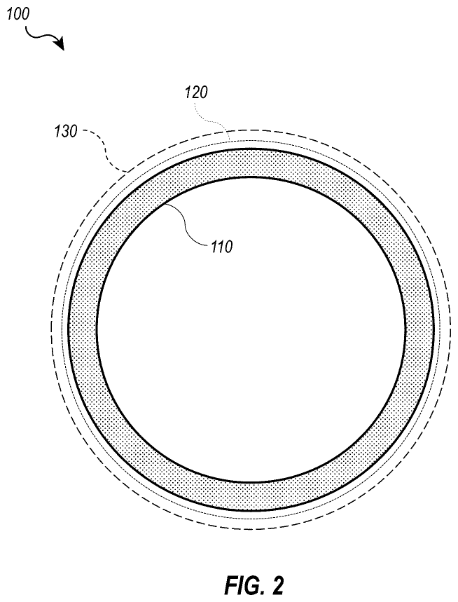

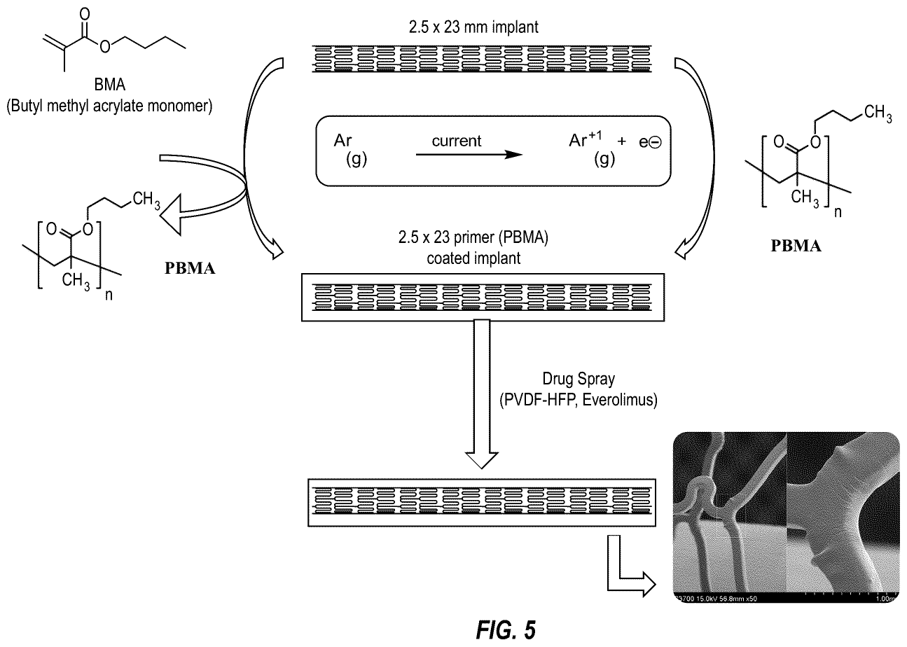

FIG. 2 illustrates a medical device or implant having a primer layer and a coating layer, according to one embodiment of the invention. As shown, the medical implant 100 includes an implant body 110 with a primer layer 120 and a coating layer 130. The implant body 110 can have the form of an expandable or self-expanding scaffolds, stent, filter, temporary implant, permanent implants, etc., with openings, interstices, etc. as illustrated in FIGS. 5-8. The primer layer 120 can be deposited on a surface of the medical implant 100 through plasma polymerization. The primer layer 120 can be formed, for example, from the monomer solution 50 illustrated in FIG. 1A. The primer layer 120 can have a thickness ranging from approximately 5 nm to approximately 0.2 μm, or approximately 5 nm to approximately 0.05 μm. For example, the primary layer 120 can have a thickness of approximately 6 nm, 8 nm, 10 nm, 12 nm, 15 nm, 20 nm, 30 nm, 40 nm, 50 nm, or a thickness within a range defined by any two of the foregoing values. Additionally, the primer layer 120 can be formed by depositing multiple layers to form the primer layer. For instance, the primer layer 120, or more generally the layer, can be formed from one layer of Poly(D,L-lactide) (PDLLA) polymer and one layer of polycaprolactone (PCL) polymer. In other configurations, the primer layer, or more generally a layer formed on the medical device, can be formed of one or more individual polymer layers.

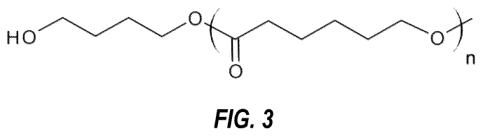

FIG. 3 illustrates one example of a monomer to be used in the primer layer 120, for example, according to one embodiment of the invention. Specifically illustrated is a caprolactone (CL) monomer. Presence of five (5) methylene groups in the CL monomer unit confers elasticity to polycaprolactone (PCL), or a polymer of the CL monomers. PCL can be constructed from any number of CL monomers, where ‘n’ represents the number of CL monomer units included in PCL. While reference is made to including of the monomer of FIG. 3 being used in the primer layer 30, it will be understood that the monomer of FIG. 3 can also be form at least a portion of the composition of the coating layer 130.

Further, the five methylene groups present in the CL monomeric unit also confers hydrophobicity to PCL, which slows the degradation kinetics of PCL and other polymers including PLLA. For example, by including PCL in the primer layer of the present disclosure, the primer layer will degrade more slowly in the body.

Other ring monomers can likewise be synthesized in a primer or other coating layer according to the present disclosure. For example, other monomers can include L-lactide, D-lactide, D,L-lactide, glycolide, caprolactone, trimethylene carbonate, p-dioxanone and/or combinations or mixtures thereof.

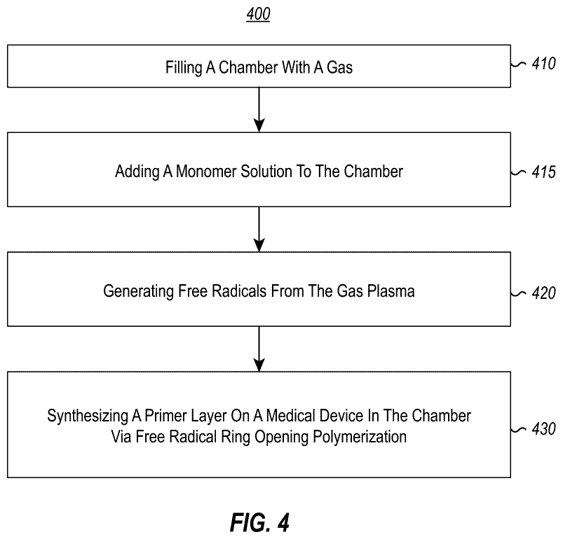

FIG. 4 illustrates a flowchart of an example method for depositing a primer layer, for example, according to one embodiment of the invention and using the system of FIGS. 1A and 1B, and any of the system illustrated and/or described in Exhibit A, which is attached hereto and incorporated herein in its entirety by the reference. FIG. 5 schematically illustrates an example of the plasma polymerization and deposition process according to the present disclosure , such as method 400.

In some embodiments, and as illustrated in FIG. 4, a method 400 for depositing a layer onto a medical implant or device. It is assumed for the method 400 that the medical implant or device is positioned on the support assembly 20 and ready to be coated. For instance, the medical implant or device can be positioned on the support assembly 20 and in preparation for coating.

Returning to FIG. 4, the method 400 can include filling a chamber with a gas or mixture of gases, such as but not limited to nitrogen, argon, etc., at step 410, in preparation for forming a gas plasma. The method 400 also includes adding a monomer solution into the chamber, at step 415. It will be noted that filling the chamber with a gas and adding the monomer solution can be performed in the same step. Once the gas and monomer solution are introduced to the chamber or reactor, activation of a power supply to generate a gas plasma to generate free radicals that interact with the monomer solution, at step 420. The method 400 further includes synthesizing a primer layer directly on a medical device contained within the chamber, at step 430. The primer layer can be synthesized via free radical ring opening polymerization.

EXAMPLES

Turning to FIGS. 5-7B and Exhibit A, examples of initial experiments using the methods and systems of the present disclosure are provided. While the experiments involved deposition of BMA monomer (n-butyl methacrylate) which polymerizes to PBMA and PBMA polymeric material (poly (butyl methacrylate)) on flat titanium coupons, the methods and systems of the present disclosure are also applicable to deposition of bioresorbable layers on medical implants or devices, such as but not limited to the bioresorbable layers described herein or otherwise contemplated by this disclosure. FIG. 5 schematically illustrates an example of the plasma polymerization and deposition process according to the present disclosure.

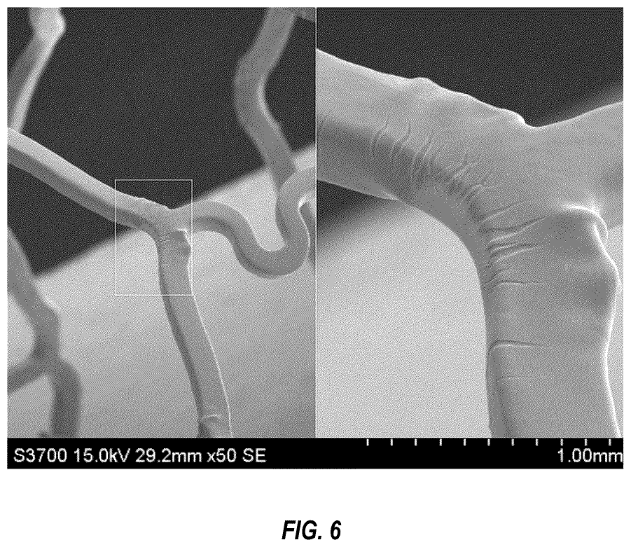

Tables 1-3 outline some of the experimental parameters. FIG. 6 illustrates a scanning electron microscope (SEM) image of a drug coating applied on top of a BMA plasma deposited metal stent in order to examine coating adhesion, according to embodiments of the present disclosure. Plasma deposition increases the water contact angle of the untreated metal) (86° to 92-98° (i.e., more hydrophobic). Surprisingly, these results are within the expected range for pure PBMA surfaces (e.g., 92-100°) (ref: Van Damme et al., J. Colloid Interf. Sci, 1986, Vol 114, pg. 167, the entire contents of which are herein incorporated by reference). This indicates that a coating has been successfully deposited, with the example Run 3 producing the highest contact angle.

During experimentation, Fourier-transform Infrared (FTIR) analysis probed the functional groups of the deposited materials (see Exhibit A). While the aliphatic hydrocarbon peaks were discernible in the 2850-3000 cm⁻¹ range, the carbonyl could not be discerned. Therefore, a longer 4-minute deposition was undertaken using the conditions from a Run 3. Longer deposition time builds up higher PBMA thickness on coupon due to presence of stronger stretch frequency. The spectra is in agreement with literature for PBMA IR.

TABLE 1

| Run | Flowrate (uL/min) | Voltage |
|---|---|---|
| 1 | 20 | 100 |
| 2 | 20 | 120 |
| 3 | 40 | 120 |
| 4 | 40 | 100 |
| controls | 0 | 0 |

TABLE 2
Equipment Settings

| | | | |
|---|---|---|---|
| BioDep | 2 | T(on) | 40 ms |
| Nebuliser | Ari Mist HP 40030 | T(off) | 1 ms |
| He | 6 L/min | Ar | 5.5 bar |
| Power | 100, 120 V | Duty | 45% |
| Frequency | 20.4 Hz | Flowrate | 20-40 uL/min |

TABLE 3

| | | | Water | | Weight |
|---|---|---|---|---|---|
| | Liquid | Plasma | Contact | Roughness | Gain |
| Coupons | Flowrate | Power (V) | Angle (°) | (Sa in nm) | (mg) |
| 1 | 20 | 100 | 86 | 85-132 | N/A |
| 2 | 20 | 120 | 92 | 99 | 0 |
| 3 | 40 | 120 | 95 | 116 | 0.02 |
| 4 | 40 | 100 | 98 | 123 | 0.03 |
| controls | 0 | 0 | 93 | 108 | 0.04 |

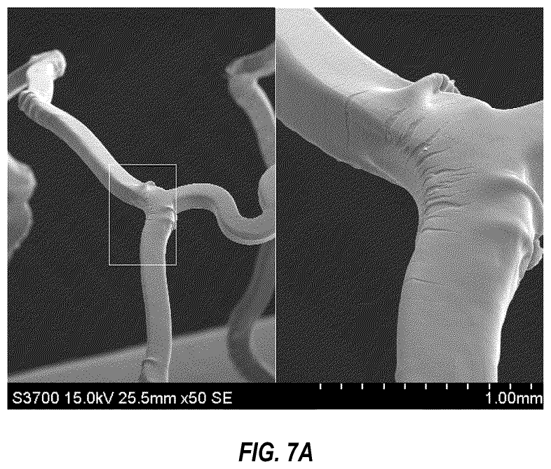

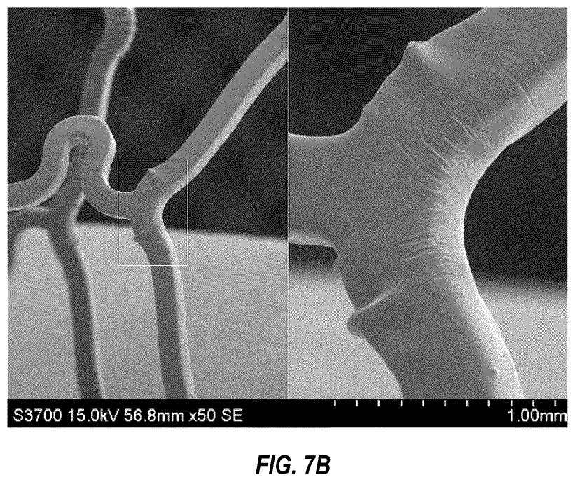

Table 4 outlines some results from a PBMA deposition experiment. FIGS. 7A-7B and Exhibit A illustrate SEM images of PBMA deposited on a substrate surface, according to embodiments of the present disclosure.

Unlike the monomer samples, these samples all displayed water contact angle values that were <90°. PBMA deposition may have undergone minor oxidation during the ionisation process thereby making the surface slightly less hydrophobic. PBMA did not alter the surface roughness of the metal and the deposited coatings all retained surface roughness values within the same range as the starting metal. 120V arms showed the greatest difference in contact angle (CA). Differences in flowrate can impact contact angle. For instance, the effect or impact on contact angle at 120V is not the same as at 100V. For instance, at 100V the contact angle does not appear to vary much as compared to the 0V contact angles, as illustrated in Exhibit A.

Again, as part of the experimentation, FTIR analysis was undertaken on the plasma deposited PBMA surface (see Exhibit A). All runs showed equivalent peak positions and intensities as the raw non-plasma treated polymer (top spectrum and black spectrum illustrated in Exhibit A). The anticipated carbonyl peak at 1727 cm⁻¹ and peaks in the fingerprint region were all present and unaltered following deposition. As the peaks were clearly defined and easily identified, this suggests that the polymeric coatings are significantly thicker than the monomer-based deposits.

TABLE 4

| | | | Water | | |
|---|---|---|---|---|---|
| | Liquid | Plasma | Contact | Roughness | Weight Gain |
| Coupons | Flowrate | Power (V) | Angle (°) | (Sa in nm) | (mg) |
| 1 | 20 | 100 | 81 | 116 | N/A |
| 2 | 20 | 120 | 73 | 102 | N/A |
| 3 | 40 | 120 | 86 | 116 | N/A |
| 4 | 40 | 100 | 82 | 124 | N/A |
| Controls | | | 83 | 85-132 | |

The deposition conditions for a Run 3 (see Table 4, above) were chosen for transfer to a stent, since the settings associated with Run 3 had produced the highest contact angle for both monomer and polymer precursors. The support assembly 20 (FIG. 1B and Exhibit A) was used during the deposition. A shroud was placed around the roller 22 (FIG. 1B) to prevent unwanted coating build-up on the rollers 22 (FIG. 1B) and to direct the gas flow onto the stent surface.

PBMA gives approximately four times higher loading of material on the surface than the corresponding BMA coating despite both processes using equivalent settings. This is due to higher vapour pressure of the BMA monomer, which allows much of the material to evaporate instead of deposition. Based on the measured weight gain and density of the polymer, it was estimated that these weight gains correspond to an average coating thickness of:
52-86 nm for the PBMA polymer, and
5-26 nm for the PBMA polymer polymerized from BMA in the plasma chamber.

Some drug eluting stents have a primer thickness of 1 micron on the outer diameter. Plasma polymerization primer layer deposition as described herein results in an approximately 11 times reduction in primer thickness relative to 86 nm thickness. These thicknesses assume a uniform coating throughout, whereas in reality, the coating may be thicker on the outer surface and thinner on the luminal surface.

The plasma deposition process was successfully developed to apply both BMA monomer and PBMA to medical implants, such as a stent. The process achieved a 52-86nm coating thickness for the PBMA polymer and a 5-26 nm coating thickness for the BMA monomer. Current commercial primer thickness is approximately 1 micron. Thus, a reduction in primer thickness of approximately 11-times (11×) was achieved using the plasma processing of the present disclosure. Drugs sprayed onto the BMA monomer (which polymerizes to PBMA during the deposition) plasma-primed units showed acceptable coating integrity, with no apparent loss of adhesion between the implant and the coating.

### Additional Terms and Definitions

The articles “a,” “an,” and “the” are intended to mean that there are one or more of the elements in the preceding descriptions. The terms “comprising,” “including,” and “having” are intended to be inclusive and mean that there may be additional elements other than the listed elements. Additionally, it should be understood that references to “one embodiment” or “an embodiment” of the present disclosure are not intended to be interpreted as excluding the existence of additional embodiments that also incorporate the recited features. Numbers, percentages, ratios, or other values stated herein are intended to include that value, and also other values that are “about” or “approximately” the stated value, as would be appreciated by one of ordinary skill in the art encompassed by embodiments of the present disclosure. A stated value should therefore be interpreted broadly enough to encompass values that are at least close enough to the stated value to perform a desired function or achieve a desired result. The stated values include at least the variation to be expected in a suitable manufacturing or production process, and may include values that are within 5%, within 1%, within 0.1%, or within 0.01% of a stated value.

A person having ordinary skill in the art should realize in view of the present disclosure that equivalent constructions do not depart from the spirit and scope of the present disclosure, and that various changes, substitutions, and alterations may be made to embodiments disclosed herein without departing from the spirit and scope of the present disclosure. Equivalent constructions, including functional “means-plus-function” clauses are intended to cover the structures described herein as performing the recited function, including both structural equivalents that operate in the same manner, and equivalent structures that provide the same function. It is the express intention of the applicant not to invoke means-plus-function or other functional claiming for any claim except for those in which the words ‘means for’ appear together with an associated function. Each addition, deletion, and modification to the embodiments that falls within the meaning and scope of the claims is to be embraced by the claims.

The terms “approximately,” “about,” and “substantially” as used herein represent an amount close to the stated amount that still performs a desired function or achieves a desired result. For example, the terms “approximately,” “about,” and “substantially” may refer to an amount that is within less than 5% of, within less than 1% of, within less than 0.1% of, and within less than 0.01% of a stated amount. Further, it should be understood that any directions or reference frames in the preceding description are merely relative directions or movements. For example, any references to “up” and “down” or “above” or “below” are merely descriptive of the relative position or movement of the related elements.

Following are some further example embodiments of the invention. These are presented only by way of example and are not intended to limit the scope of the invention in any way. Further, any example embodiment can be combined with one or more of the example embodiments.

Embodiment 1. A medical implant comprising: an implant scaffold body; a layer deposited on the implant scaffold body, the layer being a plasma polymerized polymer layer.

Embodiment 2. The medical implant of embodiment 1, wherein the layer has a thickness of about 10 nm.

Embodiment 3. The medical implant of any of embodiments 1-2, wherein the layer is bioresorbable or bioabsorbable.

Embodiment 4. The medical implant of any of embodiments 1-3, wherein the layer comprises Poly(L-lactide) (PLLA), Poly(D,L-lactide) (PDLLA), polycaprolactone (PCL), polyglycolide (PGA), poly(trimethylene carbonate), polydioxanone, or copolymers or mixtures thereof.

Embodiment 5. The medical implant of any of embodiments 1-4, wherein the layer is formed from a monomer dissolved in a solvent to form the plasma polymerized polymer layer.

Embodiment 6. The medical implant of any of embodiments 1-5, wherein the solvent comprises an aqueous solvent, an organic solvent, a liquid monomer, or combinations or mixtures thereof.

Embodiment 7. The medical implant of any of embodiments 1-6, wherein the solvent comprises caprolactone (CL), dioxanone, or combinations or mixtures thereof.

Embodiment 8. The medical implant of any of embodiments 1-7, wherein the layer comprises a polymer comprising five methylene groups in its monomer structure.

Embodiment 9. The medical implant of any of embodiments 1-8, wherein the layer comprises a ring opened polymer.

Embodiment 10. The medical implant of any of embodiments 1-9, wherein the layer is a primer layer, a coating layer formed on a primer layer, or a combination of primer layer and coating layer.

Embodiment 11. The medical implant of any of embodiments 1-10, wherein the coating layer comprises a therapeutic agent.

Embodiment 12. The medical implant of any of embodiments 1-11, wherein the therapeutic agent is everolimus, rapamycin or another olimus-based drug.

Embodiment 13. The medical implant of any of embodiments 1-12, wherein the layer comprises a primer layer, a coating layer, or combinations or mixtures thereof.

Embodiment 14. A method comprising depositing a bioresorbable layer on a surface of a medical implant, the bioresorbable layer having a thickness of about 5 nm to about 50 nm.

Embodiment 15. The method of embodiment 14, wherein the bioresorbable layer is deposited to a thickness of about 10 nm to about 20 nm.

Embodiment 16. The method of any one of embodiments 14-15, wherein the bioresorbable layer comprises Poly(L-lactide) (PLLA), Poly(D,L-lactide) (PDLLA), polycaprolactone (PCL), polyglycolide (PGA), poly(trimethylene carbonate), polydioxanone, and copolymers or mixtures thereof.

Embodiment 17. The method of any one of embodiments 14-16, wherein the bioresorbable layer comprises a polymer comprising five methylene groups in its polymer structure.

Embodiment 18. The method of any one of embodiments 14-17, wherein the bioresorbable layer comprises a plurality of layers.

Embodiment 19. The method of any one of embodiments 14-18, wherein the bioresorbable layer comprises one of Poly(D,L-lactide) (PDLLA) or polycaprolactone (PCL) or copolymer of DLLA and CL.

Embodiment 20. The method of any one of embodiments 14-19, further comprising depositing another bioresorbable layer, on the bioresorbable layer, the another bioresorbable layer comprises the other of Poly(D,L-lactide) (PDLLA), polycaprolactone (PCL), or copolymer of DLLA and CL.

Embodiment 21. The method of any one of embodiments 14-20, further comprising depositing the bioresorbable layer using plasma polymerization and/or polycondensation reactions.

Embodiment 22. The method of any one of embodiments 14-21, wherein the plasma polymerization comprises ring opening polymerization.

Embodiment 23. The method of any one of embodiments 14-22, further comprising forming a coating on the bioresorbable layer, the bioresorbable layer being a primer layer.

Embodiment 24. The method of any one of embodiments 14-23, wherein using plasma polymerization further comprises filling a chamber with a gas plasma to initiate free radical ring opening polymerization.

Embodiment 25. The method of any one of embodiments 14-24, further comprising dissolving a monomer in a solvent before depositing the bioresorbable layer on the surface of the medical implant.

Embodiment 26. The method of any one of embodiments 14-25, wherein the solvent comprises an aqueous solvent, an organic solvent, a liquid monomer, combinations or mixtures thereof.

Embodiment 27. The method of any one of embodiments 14-26, wherein the liquid monomer comprises caprolactone (CL), dioxanone or combinations or mixtures thereof.

Embodiment 28. A method of depositing a layer on a medical device, the method comprising filling a chamber with a gas plasma, wherein the medical device is contained within the chamber; adding a monomer solution into the chamber; generating free radicals configured to interact with the monomer solution; and synthesizing the layer directly on the medical device.

Embodiment 29. The method of embodiment 28, wherein the medical device is a stent.

Embodiment 30. The method of embodiment 28, wherein the medical device is a filter.

Embodiment 31. The method of any one of embodiments 27-30, wherein the monomer solution comprises one or more monomers of Poly(L-lactide) (PLLA), Poly(D,L-lactide) PDLLA, caprolactone (CL), polycaprolactone (PCL), polyglycolide (PGA), polydioxanone or poly(trimethylene carbonate).

Embodiment 32. The method of any one of embodiments 27-31, wherein the free radicals are configured to open rings of the monomers of the monomer solution.

Embodiment 33. The method of any one of embodiments 27-32, wherein the step of synthesizing the layer directly on the medical devices comprises opening ring structures of monomers within the monomer solution.

Embodiment 34. The method of any one of embodiments 27-33, where in the step of synthesizing the layer directly on the medial devices comprises polymerizing the monomers via joining of their opening ring structures.

Embodiment 35. The method of any one of embodiments 27-34, wherein the layer is a primer layer.

Embodiment 36. The method of any one of embodiments 27-35, wherein the layer comprises a therapeutic agent mixed with the layer.

Embodiment 37. The method of any one of embodiments 27-36, wherein filling the chamber with a gas plasma comprises filling the chamber with a gas or mixture of gases.

Embodiment 38. The method of any one of embodiment 27-37, wherein the gas or mixture of gases comprises at least one of nitrogen or argon.

Embodiment 39. A medical implant comprising an implant scaffold body; a primer layer deposited on the implant scaffold body, the primer layer being a plasma polymerized polymer layer and having a thickness of approximately 10 nm to approximately 20 nm, wherein the layer comprises Poly(D,L-lactide) (PDLLA) and polycaprolactone copolymer; and a coating layer applied directly on the primer layer, wherein the coating layer comprises a therapeutic agent.

Additional information and details regarding the training system of the present invention are described in the attached Appendices, which are incorporated herein by this reference. It will be understood that any of the information and details in the Appendices can be combined and/or substituted for any element, structure, features, function, etc. as described in other embodiment and configuration disclosed herein.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

1. A medical implant comprising:
an implant scaffold body;
a layer deposited on the implant scaffold body, the layer being a plasma polymerized polymer layer.

2. (canceled)

3. (canceled)

4. The medical implant of claim 1, wherein the layer comprises Poly(L-lactide) (PLLA), Poly(D,L-lactide) (PDLLA), polycaprolactone (PCL), polyglycolide (PGA), poly(trimethylene carbonate), polydioxanone, or copolymers or mixtures thereof.

5. (canceled)

6. (canceled)

7. (canceled)

8. The medical implant of claim 1, wherein the layer comprises a polymer comprising five methylene groups in its monomer structure.

9. (canceled)

10. The medical implant of claim 1, wherein the layer is a primer layer, a coating layer formed on a primer layer, or a combination of primer layer and coating layer.

11. The medical implant of claim 10, wherein the coating layer comprises a therapeutic agent.

12. The medical implant of claim 11, wherein the therapeutic agent is everolimus, rapamycin, or another olimus-based drug.

13. (canceled)

14. A method comprising:
depositing a bioresorbable layer on a surface of a medical implant, the bioresorbable layer having a thickness of about 5 nm to about 50 nm.

15. The method of claim 14, wherein the bioresorbable layer is deposited to a thickness of about 10 nm to about 20 nm.

16. (canceled)

17. The method of claim 14, wherein the bioresorbable layer comprises a polymer comprising five methylene groups in its monomer structure.

18. (canceled)

19. (canceled)

20. (canceled)

21. (canceled)

22. (canceled)

23. (canceled)

24. (canceled)

25. The method of claim 23, further comprising dissolving a monomer in a solvent before depositing the bioresorbable layer on the surface of the medical implant.

26. The method of claim 25, wherein the solvent comprises an aqueous solvent, an organic solvent, a liquid monomer, or combinations or mixtures thereof.

27. The method of claim 26, wherein the liquid monomer comprises caprolactone (CL), dioxanone or combinations or mixtures thereof.

28. A method of depositing a layer on a medical device, the method comprising:
filling a chamber with a gas plasma, wherein the medical device is contained within the chamber;
adding a monomer solution into the chamber;
generating free radicals configured to interact with the monomer solution; and
synthesizing the layer directly on the medical device.

29. The method of claim 28, wherein the medical device is at least one of a stent or a filter.

30. (canceled)

31. (canceled)

32. The method of claim 31, wherein the free radicals are configured to open rings of the monomers of the monomer solution.

33. The method of claim 28, wherein the step of synthesizing the plasma polymerized layer directly on the medical devices comprises opening ring structures of monomers within the monomer solution.

34. The method of claim 33, where in the step of synthesizing the plasma layer directly on the medial devices comprises polymerizing the monomers via joining of their opening ring structures.

35. (canceled)

36. The method of claim 28, wherein the layer comprises a therapeutic agent mixed with the layer.

37. The method of claim 28, wherein filling the chamber with a gas plasma comprises filling the chamber with a gas or mixture of gases, wherein the gas or mixture of gases comprises at least one of nitrogen or argon.

38. (canceled)

39. The medical implant of claim 1:
wherein the layer is a primer layer, the primer layer having a thickness of approximately 10 to approximately 20 nm, wherein the primer layer comprises Poly(D,L-lactide) (PDLLA), and Polycaprolactone (PCL) copolymer; and
a coating layer applied directly on the primer layer, wherein the coating layer comprises a therapeutic agent.
